(19) United States
(12) Patent Application Publication
(10) Pub. No.: US 2009/0205640 A1
(43) Pub. Date: Aug. 20, 2009

(54) APPARATUS AND METHOD FOR IMPROVING NASAL BREATHING

(76) Inventor: Gaitag Kehiaian, Los Angeles, CA (US)

Correspondence Address: Janus Law Group;Professional Law Corporation, 468 N. Camden Dr., Suite 300, Beverly Hills, CA, 90210, US

(21) Appl. No.: 12/033,805

(22) Filed: Feb. 19, 2008

(51) Int. Cl. A61M 16/00

(52) U.S. Cl. 128/200.24

(57) ABSTRACT

An apparatus and a method for improving nasal breathing are disclosed. The apparatus contains a first end, a second end, and an adhesive layer disposed on at least a portion of the first end, and a portion of the second end, wherein the first end is configured to adhere to a first skin tissue located above a bridge of a user's nose and a distance away from a tip of the user's nose, the second end is configured to adhere to a second skin tissue located above the first skin tissue, wherein the first skin tissue is pulled in a direction generally towards the user's eyes. The method disclosed teaches how to apply the apparatus.

### FIELD

The present invention relates to an apparatus and method for improving the breathing through a user's nose.

### BACKGROUND

Many individuals have difficulty breathing through their nose. Such difficulties can be the result of anatomical anomalies within the nasal passages, illnesses, allergies, etc.

Such difficulties can be detrimental to an individual's ability to sleep well. In particular, the individual may awaken frequently during the night due to breathing difficulties or may snore excessively. Moreover, breathing difficulties may also be detrimental to an individual's athletic performance, wherein it is imperative to maintain a consistent flow of air through the nasal passages and into the lungs in order to oxygenate the blood flowing therein.

In an attempt to improve breathing through the nose, numerous medications have been developed. However, such medications can be expensive. Moreover, the user can experience side effects and, in some circumstances, the user can actually develop a dependency on the medications.

In addition, surgical techniques exist to improve nasal breathing. However, such techniques are invasive and may involve an extended post-operative recovery period, and moreover may be financially burdensome to a patient.

As a result, devices 10 shown in FIGS. 1a-1d, known as nasal strips, have been created to improve breathing without the use of medications or surgical techniques. Referring to FIGS. 1a-1d, devices 10 are adhesively affixed to the nasal walls 8-9 outside of the nose 15 to gently pull the nasal walls 8-9 outwardly in the general direction of arrows 11-12, respectively, thereby enhancing the passage of air through the nose 15.

However, known nasal strips do not provide adequate retaining forces. In particular, the outward forces exerted on the nasal cartilages by known nasal strips do not hold the nasal passages sufficiently open.

### SUMMARY

Embodiments of the present disclosure provide a system and method for making a resonator.

According to a first aspect, a nose strip is disclosed, the nose strip comprising: a first end; a second end; and an adhesive layer disposed on at least a portion of the first end, and a portion of the second end; wherein the first end is configured to adhere to a first skin tissue located above a bridge of a user's nose and a distance away from a tip of the user's nose, the second end is configured to adhere to a second skin tissue located above the first skin tissue, wherein the first skin tissue is pulled in a direction generally towards the user's eyes.

According to a second aspect, a method for applying a nose strip to a user's nose is disclosed, the method comprising: selecting a nose strip comprising: a first end; a second end; an adhesive layer on at least a portion of the first end and on at least a portion of the second end; applying the first end to a first skin tissue located above a bridge of a user's nose and a distance away from a tip of the user's nose; pulling the first skin tissue in a direction generally towards the user's eyes; applying the second end to a second skin tissue located above the first skin tissue, wherein the first skin tissue is pulled in a direction generally towards the user's eyes.

Other systems, methods, features, and advantages of the present disclosure will be, or will become apparent, to one having ordinary skill in the art upon examination of the following drawings and detailed description. It is intended that all such additional systems, methods, features, and advantages included within this description, be within the scope of the present disclosure, and be protected by the accompanying claims.

### DESCRIPTION OF THE FIGURES

FIGS. 1a-1d depict nasal strips associated with PRIOR ART;

FIGS. 2-3 depict human nose;

FIGS. 4a-4b depict nose strip according to the present disclosure;

FIG. 4c depicts a side view of the nose strip according to the present disclosure;

FIGS. 4d-4f depict a method of applying nose strip according to the present disclosure;

FIG. 4g depicts a back view of the nose strip according to the present disclosure;

FIGS. 5a-5d depict other embodiments of a nose strip according to the present disclosure; and

FIGS. 6a-6c depict other methods of applying a nose strip according to the present disclosure.

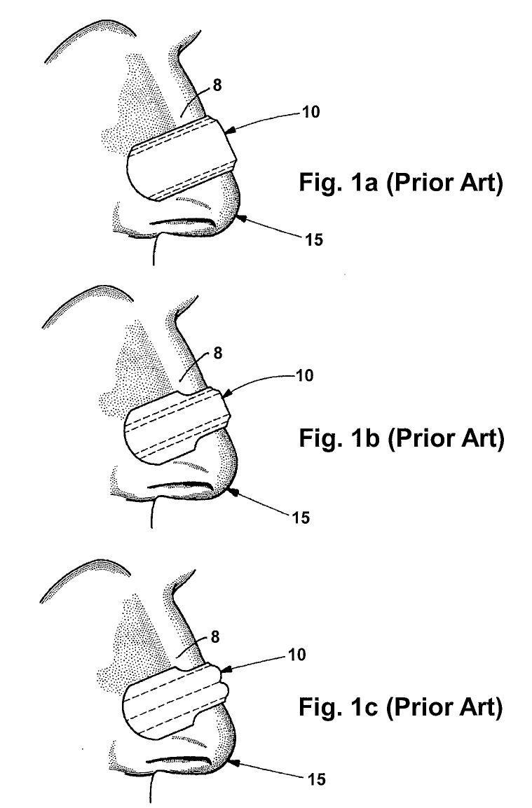

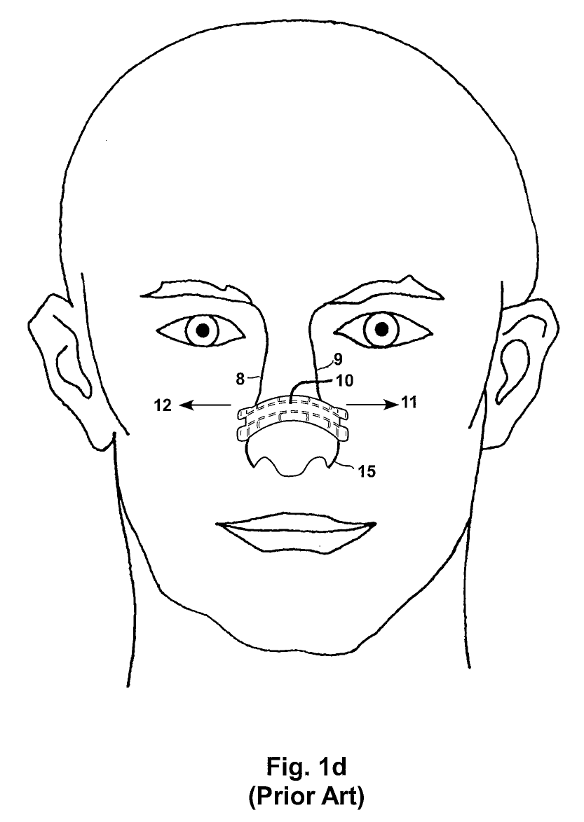

In the following description, like reference numbers are used to identify like elements. Furthermore, the drawings are intended to illustrate major features of exemplary embodiments in a diagrammatic manner. The drawings are not intended to depict every feature of every implementation nor relative dimensions of the depicted elements, and are not drawn to scale.

### DETAILED DESCRIPTION

While the invention is susceptible to various modifications and alternative forms, specific embodiments thereof have been shown by way of example in the drawings and will herein be described in detail. It should be understood, however, that there is no intent to limit the invention to the particular forms disclosed, but on the contrary, the intention is to cover all modifications, equivalents, and alternatives falling within the spirit and scope of the invention as defined by the appended claims.

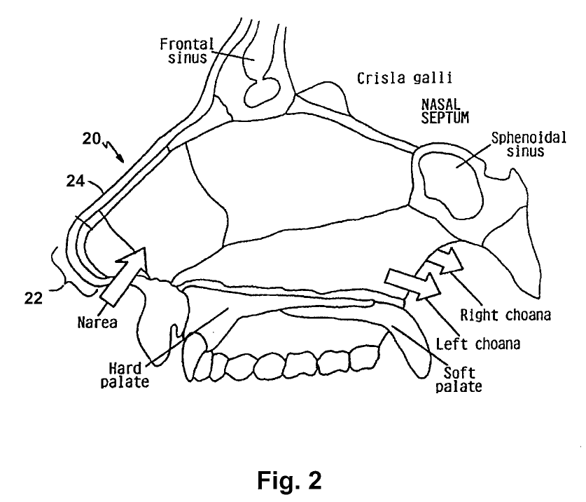

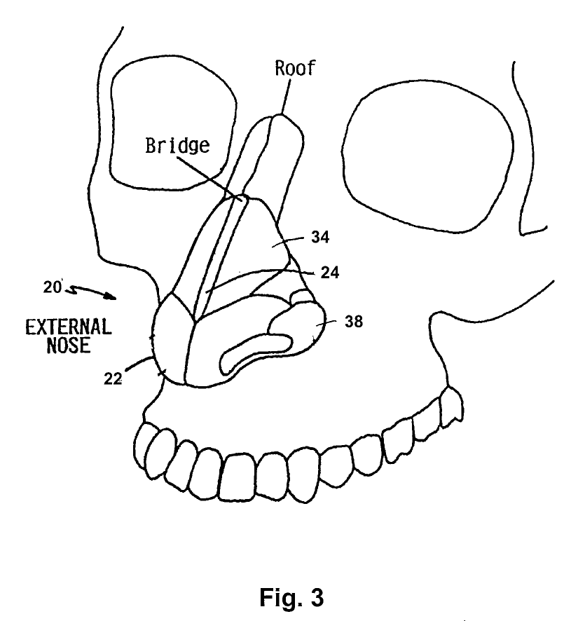

Referring now to FIGS. 2-3, there is shown a human nose 20. Nose 20 includes a tip 22 and a bridge 24. The bridge 24 of the nose 20 may generally be defined for the purposes of the present invention as running the length of the lateral nasal cartilage 34. The nose 20 further includes fibro-fatty tissue 38.

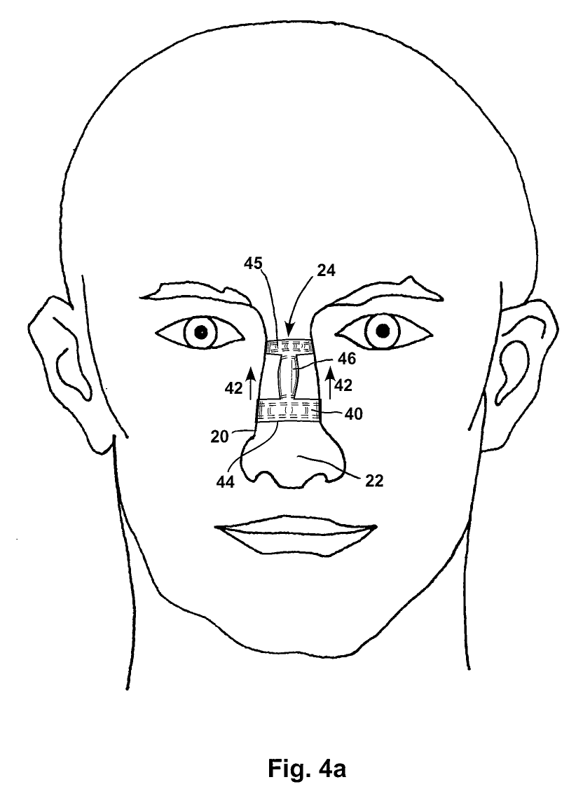

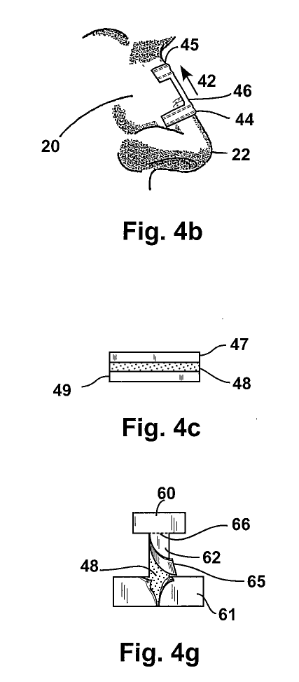

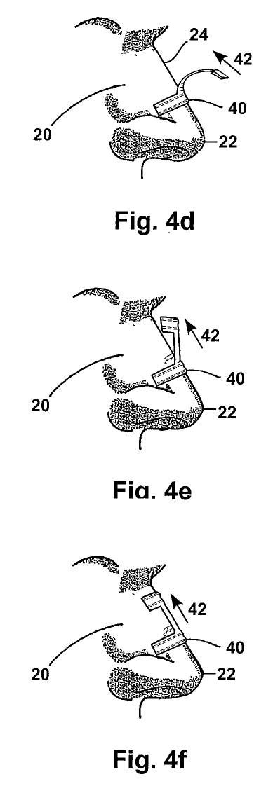

FIGS. 4a-4b depict a nose strip 40, according to the present application. The nose strip 40 may be placed along the bridge 24 of the nose 20 and away from the tip 22 of the nose 20.

The nose strip 40 may have a lower end portion 44, an upper end portion 45 and a median portion 46 connecting the lower end portion 44 and the upper end portion 45 as shown in FIGS. 4a-4b. Referring to side view FIG. 4c, the nose strip 40 may also have an upper layer 47, an adhesive layer 48 and one or more adhesive protective layers 49.

In one exemplary embodiment, the nose strip 40 may be applied by a user to the nose 20 as shown in FIGS. 4d-4f. The user may 1) peel away the one or more adhesive protective layers 49 from the nose strip 40; 2) apply the lower end portion 44 of the nose strip 40 to a skin tissue located above the bridge 24 and a distance away from nose's tip 22 as shown in FIG. 4d; 3) pull the skin tissue under the lower portion 44 in a direction 42 generally towards the eyes; 4) apply the upper portion 45 to a skin tissue located above the lower portion 44, whereby the nose strip 40 pulls the skin tissue under the lower portion 44 in a direction 42 generally towards the eyes.

In one exemplary embodiment, the upper layer 47 may be made out of, for example, an interwoven piece of fabric. The interwoven piece of fabric may have elastic properties that allow the nose strip 40 to continuously pull the skin tissue under the lower portion 44 in a direction 42 generally towards the eyes. In an exemplary embodiment, the upper layer 47 may comprise flexible nasal tape like, for example, water impervious tape (waterproof tape), fluid impermeable tape, adhesive tape, laminated tape, transparent tape, flesh colored tape, porous tape, air permeable tape, breathable tape, medical tape, or combination of the preceding. The composition of flexible tape may be made of one or more of the following: fabric, cloth, nylon, polyethylene, polypropylene, plastic, waxed fibers, composite fibers, or other flexible medical grade material.

The adhesive layer 48 may be a breathable, acrylic, nonallergenic, and/or pressure sensitive bio-compatible adhesive. The adhesive layer 48 provides a tacky bondable surface that detachably secures at least portions of the upper layer 47 to the nose and/or forehead. In one exemplary embodiment, the adhesive layer 48 is provided on a portion of the lower portion 44 and on a portion of the upper portion 45. In another exemplary embodiment, the adhesive layer 48 is provided on a portion of the lower portion 44, on a portion of the upper portion 45, and on a portion of the median portion 46.

The adhesive protective layers 49 has at least one or more removable strips 60-62 of flexible paper or plastic which detachably cover the adhesive layer 48. The removable strips may be separated by one or more cut lines 65 or perforations 66 and cooperate with the adhesive layer 48 and upper layer 47 to provide a peel and stick adhesive nasal band.

In one exemplary embodiment, the lower portion 44 of the nose strip 40 may be wider than the upper portion of the nose strip 40. In another exemplary embodiment, the upper portion 45 of the nose strip 40 is applied to the bridge 24. In yet another exemplary embodiment, the upper portion 45 of the nose strip 40 is applied to the user's forehead.

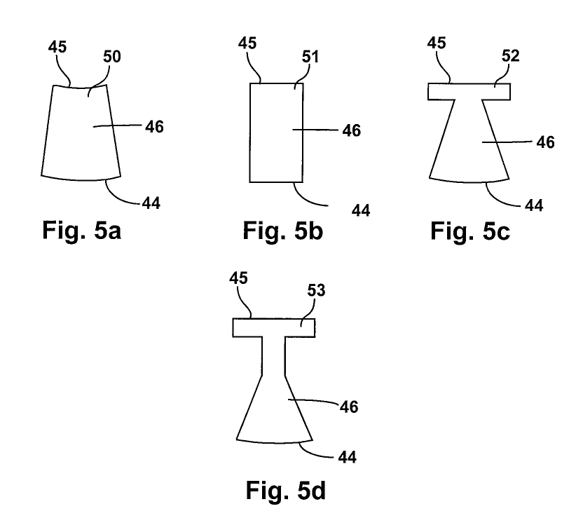

One skilled in the art would appreciate that the shape of the nose strip according to the present application is not to be limited to the shape of the nose strip 40 shown in FIG. 4a. FIGS. 5a-5d depict other exemplary embodiments, 51-53, of the nose strip according to the present application. Each of the embodiments 51-53 may also have a lower portion 44, an upper portion 45 and a median portion 46 connecting the lower portion 44 and the upper portion 45 as shown in FIGS. 5a-5d

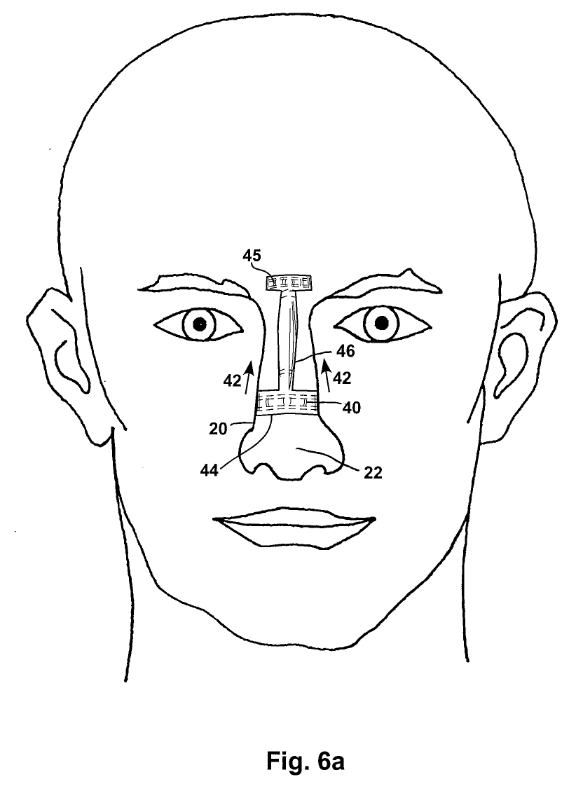

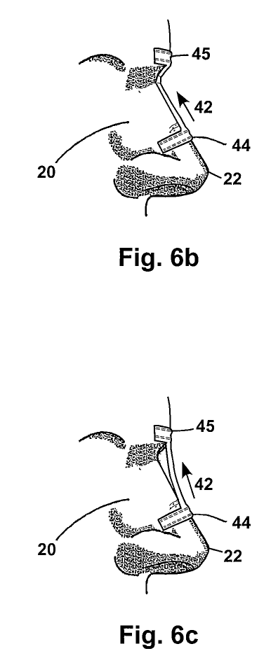

In another exemplary embodiment, the upper portion 45 of the nose strip 40 may be adhered to the user's forehead as shown in FIGS. 6a-6c. When adhesive layer is disposed on at least a portion of the median portion 46, the median portion 46 may also adhere to the bridge 24 of the nose 20 as shown in FIG. 6b. When adhesive layer is not disposed on the median portion 46, the median portion 46 may be disposed away from the bridge 24 of the nose 20 as shown in FIG. 6c.

While several illustrative embodiments of the invention have been shown and described, numerous variations and alternative embodiments will occur to those skilled in the art. Such variations and alternative embodiments are contemplated, and can be made without departing from the scope of the invention as defined in the appended claims.

As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

The foregoing detailed description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, component, or step is explicitly recited in the claims. No claim element herein is to be construed under the provisions of 35 U.S.C. Sec. 112, sixth paragraph, unless the element is expressly recited using the phrase “means for . . . ” and no method or process step herein is to be construed under those provisions unless the step, or steps, are expressly recited using the phrase “step(s) for . . . ”

What is claimed is:

1. A nose strip comprising:
a first end;
a second end; and
an adhesive layer disposed on at least a portion of the first end, and a portion of the second end;
wherein the first end is configured to adhere to a first skin tissue located above a bridge of a user's nose and a distance away from a tip of the user's nose, the second end is configured to adhere to a second skin tissue located above the first skin tissue, wherein the first skin tissue is pulled in a direction generally towards the user's eyes.

2. The nose strip of claim 1, wherein the second skin tissue is located on the bridge of the user's nose.

3. The nose strip of claim 1, wherein the second skin tissue is located on the user's forehead.

4. The nose strip of claim 1, further comprising a medial portion connecting the first end and the second end.

5. The nose strip of claim 4, wherein the adhesive layer is disposed on at least a portion of the medial portion.

6. A method for applying a nose strip to a user's nose, the method comprising:
selecting a nose strip comprising:
a first end;
a second end;
an adhesive layer on at least a portion of the first end and on at least a portion of the second end;
applying the first end to a first skin tissue located above a bridge of a user's nose and a distance away from a tip of the user's nose;
pulling the first skin tissue in a direction generally towards the user's eyes;
applying the second end to a second skin tissue located above the first skin tissue, wherein the first skin tissue is pulled in a direction generally towards the user's eyes.

7. The method of claim 6, wherein the second skin tissue is located on the bridge of the user's nose.

8. The method of claim 6, wherein the second skin tissue is located on the user's forehead.
